(19) United States
(12) Patent Application Publication
(10) Pub. No.: US 2016/0325254 A1
(43) Pub. Date: Nov. 10, 2016

(54) APPARATUS AND PROCESS FOR CATALYZED REACTIONS CARRIED OUT IN A TUBULAR REACTOR

(71) Applicant: JOHNSON MATTEY DAVY TECHNOLOGIES LIMITED, London (GB)

(72) Inventor: Julian Stuart Gray, London (GB)

(21) Appl. No.: 15/035,450

(22) PCT Filed: Jan. 29, 2015
(86) PCT No.: PCT/GB2015/050214

§ 371 Date: May 9, 2016

(30) Foreign Application Priority Data
Jan. 29, 2014 (GB) 1401518.4

(51) Int. Cl. B01J 8/06; B01J 8/02; C07C 17/08; B01J 8/00

(52) U.S. Cl. CPC B01J 8/065; B01J 8/001; B01J 8/0214; C07C 17/08; B01J 2208/00548; B01J 2208/00044; B01J 2208/00814

(57) ABSTRACT

In a reactor tube in which a plurality of catalyst carriers are disposed, each catalyst carrier is an annular container having a perforated inner wall defining a tube, a perforated outer wall, a top surface closing the annular container and a bottom surface closing the annular container. A surface closes the bottom of the tube and a skirt extends upwardly from the perforated outer wall of the annular container from a position at or near the bottom surface of the container. A seal is located at or near the top surface and extends outward from the container beyond an outer edge of the skirt. The reactor tube additionally includes a temperature measuring device configured to measure the temperature in two or more catalyst carriers within the reactor tube.

The present invention relates to an apparatus and a process for catalysed reactions carried out in a tubular reactor. The apparatus and process allow the reaction to be controlled to improve catalyst life and/or maximise catalyst productivity. More particularly, the present invention relates to an apparatus and a process which enable control of the catalysed reaction for the production of vinyl chloride.

So-called “fixed bed tubular reactors” comprise a reactor shell containing a plurality of tubes, which are usually cylindrical, and which are usually filled with catalyst particles. In use, a heat transfer means medium flows through the shell of the reactor outside these tubes and thereby adjusts the temperature of the catalyst in the tubes by heat exchange across the tube wall. Thus, where the reaction is an exothermic reaction, the heat transfer medium will allow heat to be removed from the catalyst and where the reaction is an endothermic reaction, the heat transfer medium will provide heat to the catalyst. Examples of heat transfer mediums include cooling water, boiler feed water and heat transfer oils such as that sold under the trade mark Dowtherm by The Dow Chemical Company. Alternatively the heat transfer fluid is in the form of a molten salt mixture. During operation, gas, liquid, or both gas and liquid reactants flow through the tubes over the catalyst particles such that the desired reaction takes place.

For many reactions, the heat effects of the reaction are moderate. In such circumstances large-diameter tubes may be used such that there is a large volume of catalyst within the tube. However, for more exothermic reactions it is necessary that there is efficient heat transfer via the tube wall to the heat transfer medium to enable the conditions within the reactor to be controlled. To achieve the desired efficiency, the cross-sectional area of the tube has to be reduced which means that the volume of catalyst within the tube is reduced.

Tubular reactors in which moderate to highly exothermic reactions take place are in many cases, heat transfer limited. One disadvantage of this is that the benefits of more active catalysts are difficult to realize since the increased productivity achievable with these catalysts generates increased amounts of heat which must be removed at a rate that maintains a stable operating temperature to avoid detrimental effects occurring such as side reactions taking place, damage to the catalyst such as by sintering of the catalytic active sites, and in the worst case can lead to thermal runaway. Where the reaction is a moderate to highly exothermic reaction, various problems can arise with this increased heating and in some systems the heat can be such that the catalyst can fail and even damage to the tube wall can occur.

Conventional reactors have a number of drawbacks that make them less than ideal. One problem that is noted for these reactors is that in order to extract the heat of reaction effectively the tubes have to be relatively small in diameter to ensure that the central region of the tube remains cool enough to avoid the problems detailed above occurring.

Similar, albeit converse problems, will be noted where the reaction is an endothermic reaction. In order for the heat to be provided such that the catalyst can continue to operate in the optimum manner, the tubes have to be relatively small in diameter to ensure that the central region of the tube is heated sufficiently.

In some reactions, the size restriction means that the tubes are only of the order of about 15 to 40 mm internal diameter. The small size of the tube means that, in order to accommodate the required volume of catalyst in the reactor, a large number of tubes have to be used. However, this increased number of tubes increases the weight of the reactor and since there is generally a maximum size of reactor which can be shipped in terms of dimensions and weight, the productivity of the reactor is limited.

A second problem is that the catalyst particles have to be a certain size, shape and strength so as not to cause an undue pressure drop for an appropriate tube length and in general this leads to the use of larger catalyst particles. However, the use of larger particles may be problematic where the reaction is mass or heat transfer limited, or both. Whilst some of these problems may be alleviated by ensuring that the active sites are only present near the surface of the catalyst particle, this can limit the productivity that can be achieved since the available active sites have to be worked harder to deliver a reasonable overall productivity. Whilst this may give reasonable productivity at a given time, it can reduce the life of the catalyst.

A further problem is that there is a limitation on the amount of heat that can be removed per unit surface area of the tube wall and this puts a limit on the amount of generated heat that can be tolerated per unit volume of catalyst contained within the tube.

One example of an exothermic reaction where the problems arise is the production of vinyl chloride. Vinyl chloride, which is also known as vinyl chloride monomer or chloroethane, is produced in large quantities each year. Its main use is in the production of polyvinyl chloride.

Various processes for the production of vinyl chloride have been suggested. The two main starting feedstocks are ethylene and ethyne, also known as acetylene. The choice of starting material is generally determined by the availability of raw materials to form these feedstocks in the respective locale. For example, in regions with significant coal resources, there will be access to plentiful, and hence low cost, acetylene and therefore in these regions the preferred process for the production of vinyl chloride will be to react acetylene with hydrochloric acid.

Historically, the catalyst used in the hydrochlorination of acetylene has been mercury based. However, the mercury based catalysts are highly toxic. This toxicity creates problems in loading and unloading the catalyst. There is also a risk of mercury compounds being lost into the environment and thus alternative catalysts are being sought.

One example of this new generation of mercury-free catalysts is described in WO2013/08004, the contents of which are incorporated by reference. This catalyst comprises gold on a support. Additional metals may be included in the catalyst. One suitable support is carbon although other supports can be used. Since the catalyst comprises an expensive precious metal it is important that its use is optimized.

For the new generation of catalysts to be accepted by the producers of vinyl chloride, the catalyst must perform at least as well as the conventional mercuric chloride catalyst or at least have a roughly comparable performance such that any reduction in performance is acceptable when balanced with the environmental advantages.

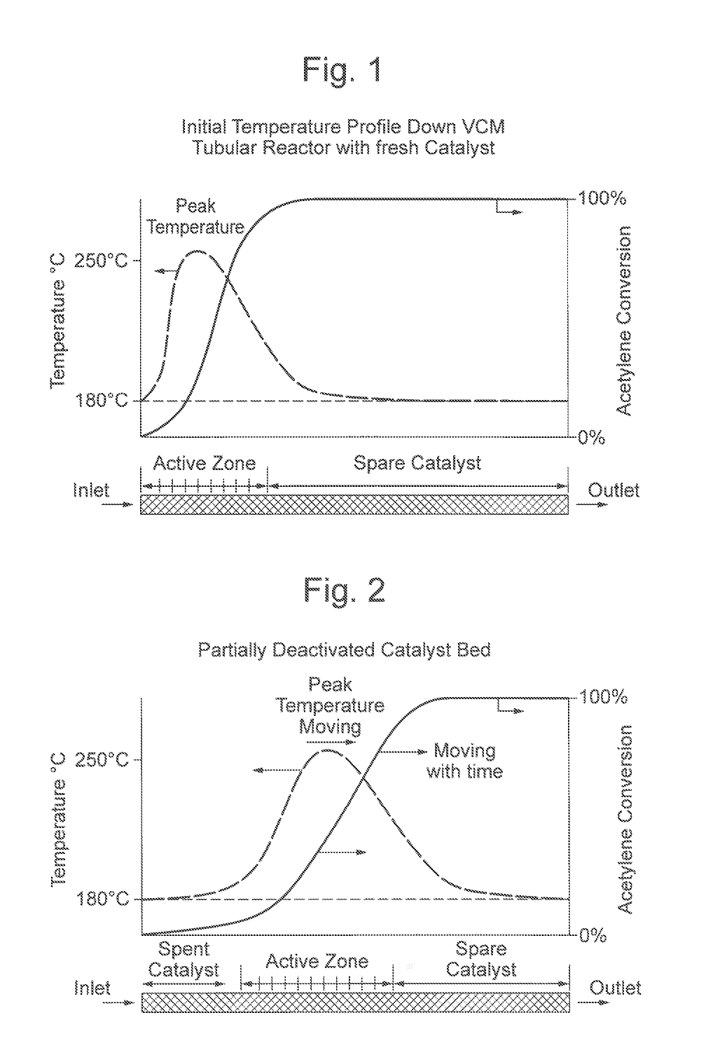

One parameter which is used for comparison of catalysts to consider their relative merits is the rate at which the catalyst ages. FIG. 1 of WO2013/08004 compares the conversion of acetylene to vinyl chloride using a conventional catalyst and the gold-based catalyst described therein and illustrates how the catalysts are deactivated with time. It will be noted that whilst fresh catalyst achieves 100% conversion, after 20 days the conversion has dropped to around 70%.

The reaction to produce vinyl chloride from acetylene, which is highly exothermic, is conventionally carried out in a fixed bed tubular reactor of the kind described above.

As in other reactions, the amount of heat generated means that only small tube diameters can be used. Although each reactor shell can include a large number of tubes, often many hundreds or even thousands, it is generally necessary for multiple reactors, often up to 100, operating in parallel, to be used in order to produce vinyl chloride at sufficient scale to meet the demands of the downstream PVC industries.

Whichever catalyst is used, it can become deactivated with time, which will result in the reduction in conversion of reactants to the desired products. This means that, in practice, sufficient catalyst is charged into the reactor to allow for the gradual deactivation of the catalyst. In this connection, it will be understood that the reference to the gradual deactivation means that initially all of the catalyst will be active but as time passes an increasing volume of the catalyst will become deactivated. Thus, for example, if one considers a 3 m tube filled with fresh catalyst, then as the feed, such as hydrochloric acid vapour and acetylene, flows into the tube, the conversion of the reactants to the product takes place over the first 5 to 10% of the tube length. In practice, the catalyst in the remainder of the tube will not be participating to any significant amount in the reaction.

As the reaction is limited to a relatively small region of the tube, the exothermic heat from the reaction is all released in quite a short section of the tube. A schematic illustration of the temperature profile at the start of the process is set out in FIG. 1.

After a short period of time, the activity of the catalyst in the first 5 to 10% of the tube starts to reduce and the reactants flow further down the tube before being converted to the desired product. Thus the active portion of the bed gradually moves down the tube until the end is reached at which point the catalyst has to be replaced. The movement of the reaction front and the peak temperature position is illustrated schematically in FIG. 2.

Since replacing the catalyst in the tubes requires the shut-down of the reactor, it is desirable that this does not occur too frequently and therefore it is common to install sufficient catalyst to enable the reactor to operate for between 6 and 12 months before the catalyst needs to be replaced.

Catalysts, including mercury catalysts and gold catalyst for use in the production of vinyl chloride, are generally sensitive to temperature and excess temperature can lead to deactivation. This is discussed further in WO2013/08004. Reactor operators therefore aim to operate their reactors in a manner to avoid excessive temperatures.

Various proposals have been made to address the problem of heat generation being high at a particular point in the reactor and of that point moving through the reactor.

One suggestion has been to vary the catalyst activity down the reactor tube either by varying the active component concentration in the catalyst or by diluting the catalyst with inerts. However, unless the catalyst deactivation can be stopped entirely the desired result is unlikely to be achieved. This is because if a low activity section is installed in the tube followed by a higher activity section, the low activity section will merely deactivate quickly and the unconverted reactants will flow rapidly through this section into the subsequent high activity section and the higher exotherm produced will rapidly deactivate this section of the bed such that the entire bed will still need to be replaced.

A further problem associated with conventional tube reactors relates to the packing of the catalyst within the tubes. This is critical to the effective functioning of the reaction. It is essential that the loading of the catalyst into the various tubes in the reactor is identical so that the pressure drop and hence the flow of reactants per tube is the same. If this does not occur, localized hot spots can occur in the tubes where the reactant flows are relatively higher. This is particularly important in highly exothermic reactions such as the production of vinyl chloride from acetylene as the formation of hot spots will exacerbate the problems discussed above.

In view of all these problems, it is desirable to operate the reactor in such a way that conditions within the reactor are controlled to keep the catalyst in the optimum operating window such that the rate of deactivation is minimised.

U.S. Pat. No. 5,759,499 describes a sealed chamber reactor for axial flow in which temperature measuring thermocouples are positioned on the wall of the tube to measure the temperature. Whilst this may give an indication of temperature within the reactor it does not give a direct measure of the actual temperature.

In order to control the reactor to achieve the optimum operating window, it is necessary to know the peak temperature being achieved within the reactor. This is because it is the peak temperature which determines the rate at which the catalyst deactivates. However, in practice it is very difficult to measure the actual peak temperature in a bed of catalyst particles and the temperature in the centre of the tube can be 20 to 50° C. hotter than the temperature at the tube wall where heat is being removed by cooling by exchange with the cooling medium at the tube wall. Thus, measurement techniques such as the one described in U.S. Pat. No. 5,759,499 which rely on temperature measurements made at the wall of the tube are not suitable for obtaining peak temperature measurements.

Further, even if it is attempted to measure the temperature in the centre of the tube, there is no guarantee that the temperature measured is, in fact, the peak temperature. Factors such as whether the thermocouple tip is in direct contact with a catalyst particle or not can affect the reading. A further problem is that in a tubular reactor, whilst the gas flows axially, the heat flow within the tube is perpendicular to the gas flow, i.e. radially. This means that mixing effects and variations in catalyst loading within the tube all affect the temperature measurement.

An alternative arrangement is described in U.S. Pat. No. 6,657,088 where a system for measuring temperature within a tubular reactor is described. Two specific constructions are described. In the first design, the thermocouple is fitted axially displaceably and centrally in the tubular reactor and the temperature profile is measured by axial displacement. However, a thermocouple which is axially displaceable within a reactor tube is difficult to engineer.

In the second design, multiple elements are used whose measuring points are disposed at different axial positions in the tubular reactors, so that they provide information on the temperature profile along the tubular reactor. One problem associated with this design is that it requires sufficient headspace above the tubular reactor for the thermocouple to be raised from the reactor tube. Given that reactor tubes can be in excess of 12 m long, this means that if the thermocouple is required to measure temperature at the bottom of the reactor tube, over 12 m of headspace may be required above the reactor tube. This would be difficult to achieve in practice and is therefore unfavourable.

In any event, both designs share a common problem in that the temperature measurement means would not measure the peak temperature. The temperature measurement means of both designs is likely to cause heat dissipation, meaning the temperature measured would not be the peak temperature particularly since there is no control over whether the tip of the thermocouple is in direct contact with catalyst particles or not.

A further problem is that the presence of the temperature measurement means interferes with the gas flow and in effect will form a thermowell. One consequence of this is the formation of a boundary layer flow on the surface of the temperature measuring means. The temperature of the boundary layer will differ from the temperature within the catalyst bed and as such, the temperature measured would not be the peak temperature of the catalyst bed. Further, the existence of the thermowell may conduct heat away from the hottest point in the reactor in an axial direction. It will therefore be understood that the accuracy of any temperature measurement in the axial tube reactor can be considered to be limited.

Similar difficulties are noted where the reaction is an endothermic reaction.

In WO2011/048361 an alternative approach to packing catalyst in tubes is discussed. In particular, a catalyst carrier device is described which is configured to sit within the reactor tube and which in use optimises heat transfer at the tube wall such that larger tubes and larger volumes of smaller catalyst particles can be used. This arrangement, allows the reactor to be operated at high productivity and an acceptable pressure drop even where the reaction is highly exothermic.

The catalyst carrier described in WO 2011/048361 is configured for insertion in a tube of a tubular reactor. The catalyst carrier comprises:

an annular container for holding catalyst in use, said container having a perforated inner wall defining a tube, a perforated outer wall, a top surface closing the annular container and a bottom surface closing the annular container;

a surface closing the bottom of said tube formed by the inner wall of the annular container;

a skirt extending upwardly from the perforated outer wall of the annular container from a position at or near the bottom surface of said container to a position below the location of a seal;

and a seal located at or near the top surface and extending from the container by a distance which extends beyond an outer surface of the skirt.

Whilst these catalyst carriers offer various advantages, it is still desirable to be able to control the reactor to maintain the catalyst in the optimum operating window.

Where these catalyst carriers are used, the heat of the reaction and the gas flow both travel in the same direction, i.e. radially through the catalyst bed. This means that the peak temperature of any exothermic reaction carried out using the carrier will be at the outer edge of the carrier. This is in direct contrast to the situation with conventional tube reactors the temperature flow path of which is discussed above.

It is therefore possible to include temperature measuring apparatus within the catalyst carrier to provide an accurate indication of the peak temperature under which the catalyst is operating. If a true peak temperature measurement can be obtained, then the reaction conditions can be better controlled to ensure that the catalyst is kept in the optimum operating window such that the rate of catalyst deactivation is minimised.

Thus, according to the present invention there is provided a reactor tube comprising a plurality of catalyst carriers, each of said catalyst carriers comprising:

an annular container for holding catalyst in use, said container having a perforated inner wall defining a tube, a perforated outer wall, a top surface closing the annular container and a bottom surface closing the annular container;

a surface closing the bottom of said tube formed by the inner wall of the annular container;

a skirt extending upwardly from the perforated outer wall of the annular container from a position at or near the bottom surface of said container to a position below the location of a seal;

a seal located at or near the top surface and extending from the container by a distance which extends beyond an outer surface of the skirt;

characterised in that the reactor tube additionally includes a temperature measuring device configured to measure the temperature in two or more catalyst carriers.

It will therefore be understood that in this arrangement, the temperature measuring device is in contact with well mixed gas at a point where there is effectively no reaction taking place and no heat transfer and as such a more accurate temperature measurement is achieved. Further, since the reaction bed is adiabatic, the measurement will give an accurate measurement of the peak temperature.

In one arrangement, the temperature measuring device measures the temperature in substantially the same position in each of the catalyst carriers in which the temperature is to be measured. This may include being located at the same height within each carrier in which the temperature is to measured.

For the avoidance of doubt, any discussion of orientation, for example terms such as upwardly, below, lower, and the like have, for ease of reference been discussed with regard to the orientation of the catalyst carrier as illustrated in the accompanying drawings. However, the catalyst carrier of the present invention could also be used in an alternative orientation for example horizontally. Thus the terms should be constructed accordingly. The container will generally be sized such that it is of a smaller dimension than the internal dimension of the reactor tube into which it is to be placed in use. The seal will be sized such that it interacts with the inner wall of the reactor tube when the catalyst carrier of the present invention is in position within the tube. Parameters such as carrier length and diameter will be selected to accommodate different reactions and configurations.

In use in a vertical reactor with downflow, reactant(s) flow downwardly through the tube and thus first contacts the upper surface of the catalyst carrier. Since the seal blocks the passage of the reactant(s) around the side of the container, the top surface thereof directs them into the tube defined by the inner perforated wall of the container. The reactant(s) then enters the annular container through the perforated inner wall and then passes radially through the catalyst bed towards the perforated outer wall. During the passage from the inner wall to the outer wall, the reactant(s) contact the catalyst and reaction occurs. Unreacted reactant and product then flow out of the container though the perforated outer wall. The upwardly extending skirt then directs reactant and product upwardly between the inner surface of the skirt and the outer wall of the annular container until they reach the seal. They are then directed, by the underside of the seal, over the end of the skirt and flow downwardly between the outer surface of the skirt and the inner surface of the reactor tube where heat transfer takes place. It will be understood that where the reactor is an upflow reactor or is for example in a horizontal orientation, the flow path will differ from that described above. However the principle of the path through the container will be as described.

Any suitable temperature measuring means may be used. However, it will be need to be selected to withstand the operating conditions within the reactor.

The temperature measuring means may be located at any suitable place within the carrier. If the temperature of the gas exiting the catalyst bed is measured in the area of the carrier between the catalyst bed and the skirt, or outside of the skirt, the measured temperature will be an accurate indication of the peak temperature under which the catalyst is operating. In order to measure the peak temperature, it is essential that the temperature measurement is made before heat transfer takes place.

It will be understood that the catalyst carrier can be used in a reactor where the flow direction is reversed. In this arrangement, the reactants flow from the outside of the catalyst bed into the centre of the annular container and then flow upwardly and out to the next container where the temperature of the gas is altered before entering the next catalyst bed.

In the arrangement in which the flow is reversed, the temperature measuring means may be located in the space in the centre of the annular container. In this embodiment, the centrally located thermocouple will measure the peak temperature at the exit of the catalyst bed.

Generally, a plurality of catalyst carriers will be stacked within a reactor tube. In this arrangement, when operated with conventional flow, the reactants/products flow downwardly between the outer surface of the skirt of a first carrier and the inner surface of the reactor tube until they contact the upper surface and seal of a second carrier and are directed downwardly into the tube of the second carrier defined by the perforated inner wall of its annular container. The flow path described above is then repeated. Where a plurality of carriers is used, a temperature measuring means may be present in each carrier. In one alternative arrangement, a temperature measuring means may be present in a selection of the carriers.

The temperature measuring device configured to measure the temperature in two or more catalyst carriers may be a multipoint thermocouple. The same temperature measuring device may be used to measure the temperature in all of the catalyst carriers in which the temperature is to be measured. Alternatively, more than one temperature measuring device may be used provided that each is used to measure the temperature in two or more catalyst carriers. Where there are a plurality of tubes present, temperature measuring means may not be present in carriers in every tube but rather in one or a selection thereof.

Where a multipoint thermocouple is used, any suitable arrangement may be used. One arrangement is the multipoint sensor available from Rosemont. Temperature may be measured in every catalyst carrier in the tube or in a selection of carriers. The selection of the carriers in which the temperature is to be measured, may vary in different parts of the tube. Thus, for example, depending on the reaction to be conducted, and the temperature profile of the reactor, it may be desirable to measure the temperature in every catalyst carrier in one part of the tube and in fewer catalyst carriers in another part of the tube.

The temperature measuring means may be installed in position in the tube before or after the catalyst carriers are placed in the tube. Where the temperature measuring means is located in position before the tube is loaded with catalyst carriers, the temperature measuring means may be used to facilitate the alignment of the catalyst carriers.

Where the temperature measuring means is to be inserted after the tube has been loaded with catalyst carriers, the catalyst carrier may have a region shaped to assist the user to guide the temperature measuring means into the catalyst carrier. Any suitable shaping which allows the temperature measuring means to be guided may be used. However, a conical configuration may be advantageous.

The catalyst carrier may additionally include shaping at the point of exit of the thermocouple which in use will serve to guide the thermocouple as it exits one catalyst carrier and direct it in the correct orientation for entry to the next carrier.

Thus the invention also relates to a method of loading a reactor tube comprising one of: a process for loading catalyst carriers into a reactor tube comprising inserting the temperature measuring means into the reactor tube and then loading the catalyst carriers over the temperature means; or a process for loading catalyst carriers into a reactor tube comprising inserting the catalyst carriers into the reactor tube and then feeding the temperature measuring means into the carriers.

Whichever temperature measuring means is used and wherever it is located within the carrier, appropriate mounting means and seals may be provided to ensure that the presence of the temperature measuring device does not perturb the gas flow path through the carrier and hence through the tube.

The catalyst carrier may be formed of any suitable material. Such material will generally be selected to withstand the operating conditions of the reactor. Generally, the catalyst carrier will be fabricated from carbon steel, aluminium, stainless steel, other alloys or any material able to withstand the reaction conditions.

The wall of the annular container can be of any suitable thickness. Suitable thickness will be of the order of about 0.1 mm to about 1.0 mm, preferably of the order of about 0.3 mm to about 0.5 mm.

The size of the perforations in the inner and outer walls of the annular container will be selected such as to allow uniform flow of reactant(s) and product(s) through the catalyst while maintaining the catalyst within the container. It will therefore be understood that their size will depend on the size of the catalyst particles being used. In an alternative arrangement the perforations may be sized such that they are larger but have a filter mesh covering the perforations to ensure catalyst is maintained within the annular container. This enables larger perforations to be used which will facilitate the free movement of reactants without a significant loss of pressure.

It will be understood that the perforations may be of any suitable configuration. Indeed where a wall is described as perforated all that is required is that there is means to allow the reactants and products to pass through the walls. These may be small apertures of any configuration, they may be slots, they may be formed by a wire screen or by any other means of creating a porous or permeable surface.

Although the top surface closing the annular container will generally be located at the upper edge of the or each wall of the annular container, it may be desirable to locate the top surface below the upper edge such that a portion of the upper edge of the outer wall forms a lip. Similarly, the bottom surface may be located at the lower edge of the, or each, wall of the annular container or may be desirable to locate the bottom surface such that it is above the bottom edge of the wall of the annular container such that the wall forms a lip. The bottom surface of the annulus and the surface closing the bottom of the tube may be formed as a single unit or they may be two separate pieces connected together. The two surfaces may be coplanar but in a preferred arrangement, they are in different planes. In one arrangement, the surface closing the bottom of the tube is in a lower plane than the bottom surface of the annular container. This serves to assist in the location of one carrier on to a carrier arranged below it when a plurality of containers are to be used. It will be understood that in an alternative arrangement, the surface closing the bottom of the tube may be in a higher plane that the bottom surface of the annular container.

Whilst the bottom surface will generally be solid, it may include one or more drain holes. Where one or more drain holes are present, they may be covered by a filter mesh. Similarly a drain hole, optionally covered with a filter mesh may be present in the surface closing the bottom of the tube. Where the carrier is to be used in a non-vertical orientation, the drain hole, where present will be located in an alternative position i.e. one that is the lowest point in the carrier when in use.

One or more spacer means may extend downwardly from the bottom surface of the annular container. The, or each, spacer means may be formed as separate components or they may be formed by depressions in the bottom surface. Where these spacer means are present they assist in providing a clear path for the reactants and products flowing between the bottom surface of the first carrier and the top surface of a second lower carrier in use. The spacer may be of the order of about 4 mm to about 6 mm deep. Alternatively, or additionally, spacer means may be present on the top surface.

The top surface closing the annular container may include on its upper surface means to locate the container against a catalyst carrier stacked above the container in use. The means to locate the container may be of any suitable arrangement. In one arrangement it comprises an upstanding collar having apertures or spaces therein to allow for the ingress of reactants.

The upwardly extending skirt may be smooth or it may be shaped. Any suitable shape may be used. Suitable shapes include pleats, corrugations, and the like. The pleats, corrugations and the like will generally be arranged longitudinally along the length of the carrier. The shaping of the upstanding skirt increases the surface area of the skirt and assists with the insertion of the catalyst carrier into the reaction tube since it will allow any surface roughness on the inner surface of the reactor tube or differences in tolerances in tubes to be accommodated.

Where the upwardly extending skirt is shaped, it will generally be flattened to a smooth configuration towards the point at which it is connected to the annular container to allow a gas seal to be formed with the annular container. The upstanding skirt will generally be connected to the outer wall of the annular container at or near the base thereof. Where the skirt is connected at a point above the bottom of the wall, the wall will be free of perforations in the area below the point of connection. The upstanding skirt may be flexible. Generally, the upstanding skirt will stop at about 0.5 cm to about 1.5 cm, preferably about 1 cm, short of the top surface of the annular container.

Without wishing to be bound by any theory, it is believed that the upstanding skirt serves to gather the reactants/products from the perforated outer wall of the annular container and direct them via the shapes towards the top of the catalyst carrier collecting more reactants/products exiting from the outer wall of the annular container as they move upwardly. As described above, reactants/products are then directed down between the tube wall and the outside of the upstanding skirt. By this method the heat transfer is enhanced down the whole length of the carrier but as the heat exchange is separated from the catalyst, hotter or colder as appropriate heat exchange fluid can be used without quenching the reaction at the tube wall and at the same time ensuring that the temperature of the catalyst towards the centre of the carrier is appropriately adjusted.

The seal may be formed in any suitable manner. However, it will generally be sufficiently compressible to accommodate the smallest diameter of the reactor tube. The seal will generally be a flexible, sliding seal. In one arrangement, an O-ring may be used. A compressible split ring or a ring having a high coefficient of expansion could be used. The seal may be formed of any suitable material provided that it can withstand the reaction conditions. In one arrangement, it may be a deformable flange extending from the carrier. The flange may be sized to be larger than the internal diameter of the tube such that as the container is inserted into the tube it is deformed to fit inside and interact with the tube.

The catalyst can be provided to the user within the carriers of the present invention which can then be readily installed within the reactor tubes with minimum downtime. Thus catalyst may be loaded into the catalyst carrier at the catalyst manufacturing site. If the catalyst requires any pretreatment before it is used in the desired reaction, such as the reduction of metal species in the catalyst to a lower oxidation states or stabilisation of the catalyst under inert atmospheres, this can be done when the containers are filled with the catalyst obviating the need for catalyst handling on site. Once the catalyst is spent, the carriers can be readily removed from the reactor as discrete units and readily transported for disposal or regeneration as appropriate. This is particularly advantageous where the catalyst is toxic, such as when the mercuric catalysts are used for the production of vinyl chloride. It also offers advantages where the catalyst comprises precious metals, such as the gold-based catalyst used in the production of vinyl chloride, since the loss of valuable material to third parties can be prevented.

A further advantage of the present invention is that the problems noted in prior art arrangements in ensuring that each tube of a tubular reactor are equally filled are obviated. The catalyst carrier of the present invention allows the use of highly granular or structured catalysts in medium to highly exothermic or endothermic reactions. The device allows the use of large tubes leading to large weight and cost reductions for a reactor of a given capacity since heat transfer effectively takes place in a micro-channel zone at the tube wall. This gives excellent heat transfer to or from the cooling/heating medium. Furthermore, as the catalyst is separated from the cooling/heating medium, a larger temperature difference can be allowed as the heat exchange effect is separated from the reaction. Where a plurality of carriers of the present invention is inserted into a tube this effectively provides a plurality of adiabatic reactors in series in each tube.

The catalyst carrier of the present invention may be filled or partially filled with any suitable catalyst. The selection of the catalyst will depend on the reaction to be carried out.

Where the reaction to be carried out is the production of vinyl chloride, the catalyst chosen will be one suitable for that reaction. Thus the catalyst may be a mercuric catalyst or a gold-based catalyst, and in particular may be the catalyst described in WO2013/08004. Thus the catalyst may comprise a complex of gold with a sulphur-containing ligand on a support. The sulphur-containing ligand may be an oxidising ligand containing sulphur in a positive oxidation state. In one arrangement, the sulphur-containing ligand may be derived from a compound selected from the group consisting of a sulphonate, thiosulphate, a thiocyanate, thiourea, thionyl chloride, thiopropionic acid and thiomalic acid.

In one arrangement, the catalyst may comprise gold, or a compound thereof, and trichloroisocyanuric acid or a metal dichloroisocyanurate on a support. The support may comprise carbon and/or a metal oxide. The support may be in the form of a powder, granulate or shaped unit.

At least some of the gold may be in a positive oxidation state. The catalyst may further comprise a metal or a compound of a metal selected from the group consisting of cobalt, copper, lanthanum, cerium, lithium, sodium, potassium, rubidium, caesium, magnesium, calcium, strontium and barium.

According to a second aspect of the present invention there is provided a reactor comprising one or more of the reactor tubes of the above first aspect.

According to a third aspect of the present invention there is provided a process for controlling a reaction comprising the steps of:

supplying reactants to a reactor of the above second aspect such that they come into contact with the catalyst in the carrier;

allowing the reaction to occur;

monitoring the temperature measured by the temperature measuring means; and

adjusting a parameter of the reactor when the temperature measured by the temperature means deviates from a predetermined range.

By measuring the temperature in two or more of the carriers of the present invention, the operator can adjust a variety of parameters to, for example, minimise catalyst deactivations discussed above. In addition, the information received from the temperature measuring means may enable the operator to adjust the operation of the reactor, for example, to control the reaction rate, to minimise the formation of by-products, or to maximise conversion of feedstock.

Any suitable parameter of the reactor can be adjusted that provides the desired results. For example, the flow rate of one or more reactants may be controlled in order to adjust the reaction rate and hence how much heat is generated, or, in an endothermic reaction, released. Additionally, or alternatively, the temperature and/or flow rate of the heat transfer medium in order to alter the temperature difference across the tube wall and hence adjust the heat transfer rate across the tube wall.

The catalyst carrier of the present invention may be used in a wide range of processes. Examples of suitable processes include reactors for exothermic reactions such as reactions for the production of methanol, reactions for the production of vinyl chloride, ammonia, methanation reactions, shift reactions, oxidation reactions such as the formation of maleic anhydride and ethylene oxide, Fischer-Tropsch reactions, and the like. They may also be used in endothermic reactions such as pre-reforming, dehydrogenation and the like where it may also be appropriate to monitor the temperature profile within the reactor.

A further advantage of the present invention is that by being better able to measure the actual reactor operating temperature within the catalyst beds, the reactor operating conditions can be adjusted to keep the catalyst temperature in the optimum region and as discussed above, this will lead to a reduction in the rate of catalyst deactivation. In the reaction to produce vinyl chloride, the reactor operating conditions can be adjusted to keep the catalyst temperature in the region of 150° C. and 200° C. Without wishing to be bound by any theory, it is believed that every 10° C. reduction in catalyst operating temperature, reduces the rate of catalyst ageing by 15 to 20%. It will however be understood that there is a limit to the reduction of the temperature which can be used as it will also slow the reaction and may even call it to stop. This means that either less catalyst can be installed within a reactor to achieve the same bed lifetime or, if the same catalyst volume is installed, a longer bed lifetime can be achieved. It is believed that this is because a greater percentage of the installed catalyst volume is involved in the reaction at any moment in time. Thus, in the production of vinyl chloride, it might be that 10% of the installed catalyst is actually converting acetylene to vinyl chloride at any moment in time. It should be understood that this percentage is comparing catalyst in a conventional reactor of an equivalent age.

It will be understood that there is normally an optimum window of operation, too high a temperature and whilst the reaction rate will be faster so more product is produced per catalyst unit volume, the catalyst ages faster and so either more catalyst volume is necessary or more frequent changes of catalyst will be required. However, if the reactor is operated at too low a temperature, the rate of ageing will be reduced so that the required catalyst changes will less frequent but the reaction rate per unit catalyst volume will be so slow that the required catalyst volume and the reactor will be very large. It is therefore desirable to operate the reactor at the most overall economic performance.

The present invention will now be described, by way of example, with reference to the accompanying drawings in which:

FIG. 1 is a schematic illustration of the temperature profile at the start of the process in a conventional tube;

FIG. 2 is a schematic illustration of the movement of the reaction front and the peak temperature position in a convention tube;

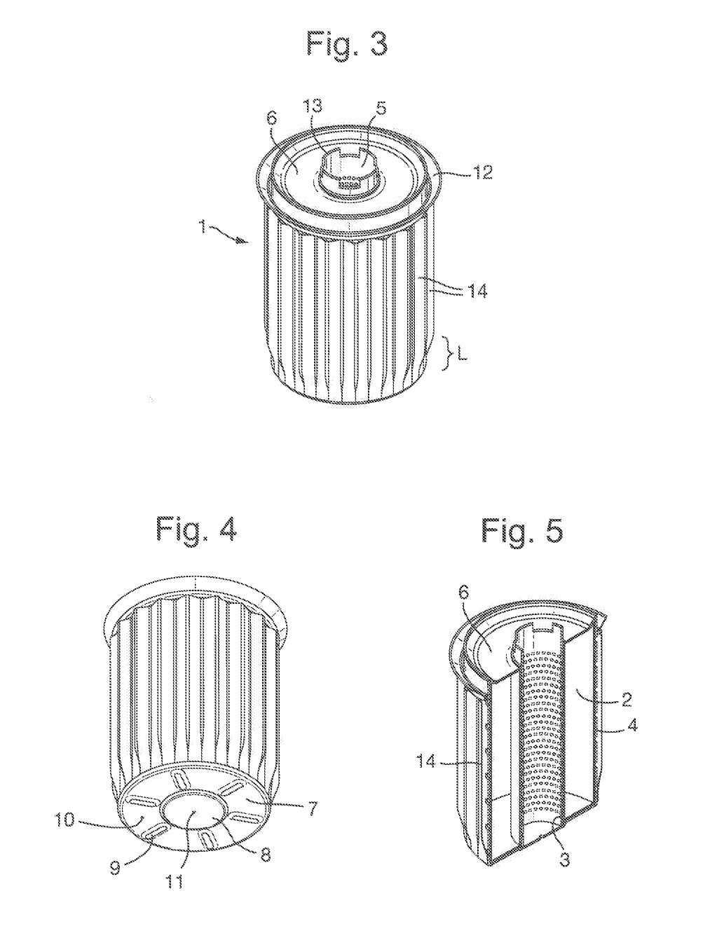

FIG. 3 is a perspective view from above of the catalyst carrier of the present invention (temperature measuring means omitted);

FIG. 4 is a perspective view of the catalyst carrier from below (temperature measuring means omitted);

FIG. 5 is a partial cross section viewed from the side (temperature measuring means omitted);

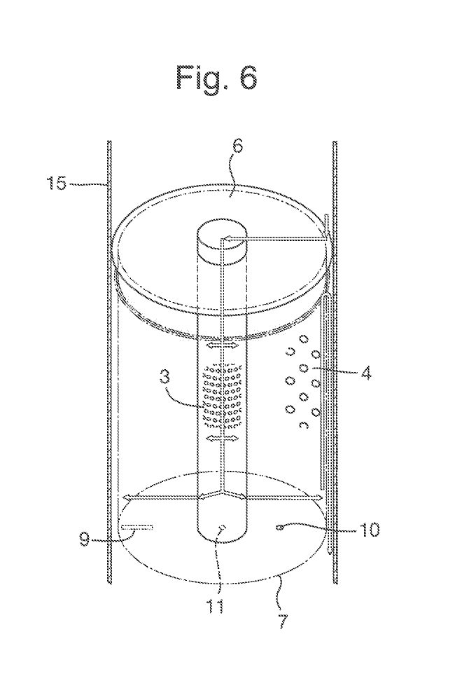

FIG. 6 is a simplified diagram of the catalyst carrier of the present invention (temperature measuring means omitted);

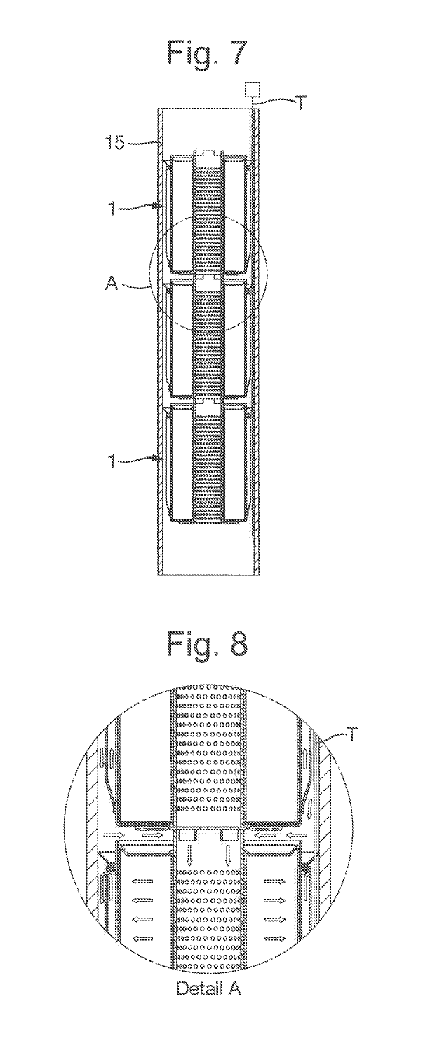

FIG. 7 is a schematic cross section of three catalyst carriers located within a tube illustrating one position of the temperature measuring means;

FIG. 8 is an enlarged cross-section of Section A of FIG. 7;

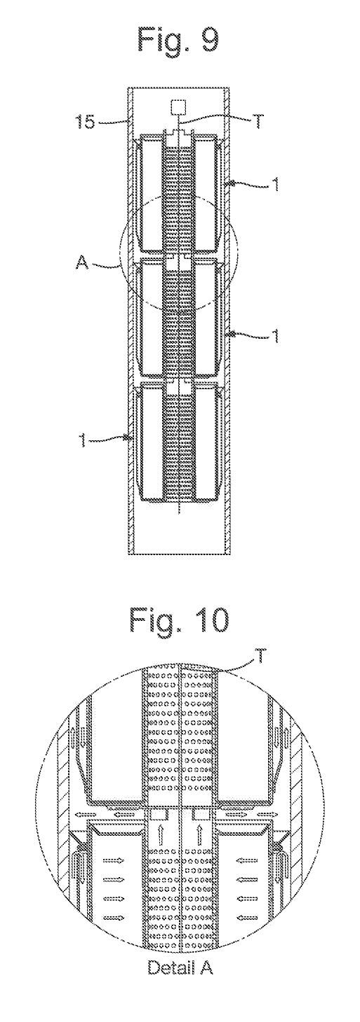

FIG. 9 is a schematic cross section of three catalyst carriers located within a tube having a reversed direction of flow, illustrating an alternative position of the temperature measuring means;

FIG. 10 is an enlarged cross-section of Section A of FIG. 9; and

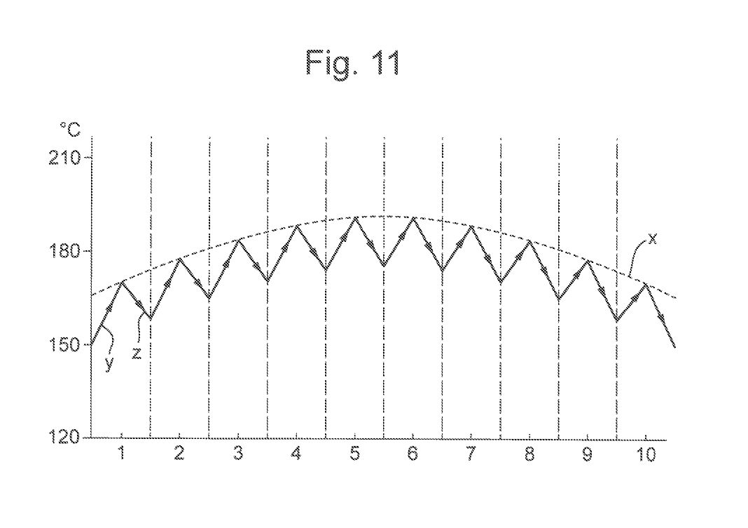

FIG. 11 is a schematic representation of the temperature measurement profile in the present invention.

A catalyst carrier 1 of the present invention is illustrated in FIGS. 3 to 5. For clarity the temperature measuring means has been omitted. The carrier 1 comprises an annular container 2 which has perforated walls 3, 4. The inner perforated wall 3 defines a tube 5. A top surface 6 is closes the annular container at the top. It is located at a point towards the top of the walls 3, 4 of the annular container 2 such that a lip 6 is formed. A bottom surface 7 closes the bottom of the annular container 2 and a surface 8 closes the bottom of tube 5. The surface 8 is located in a lower plane that that of the bottom surface 7. Spacer means in the form of a plurality of depressions 9 are located present on the bottom surface 7 of the annular container 2. Drain holes 10, 1 1 are located on the bottom surface 7 and the surface 8.

A seal 12 extends from the upper surface 6 and an upstanding collar 13 is provided coaxial with the tube 5.

A corrugated upstanding skirt 14 surrounds the container 2. The corrugations are flattened in the region L towards the base of the carrier 1.

A catalyst carrier 1 of the present invention located in a reactor tube 15. The flow of gas is illustrated schematically in FIG. 6 by the arrows.

When a plurality of catalyst carriers of the present invention are located within a reactor tube 15 they interlock as illustrated in FIGS. 7 and 8. The flow path is further illustrated in the enlarged section shown in FIG. 8 by the arrows.

In one arrangement, as illustrated in FIGS. 7 and 8 the temperature measuring means may be a thermowell located between the skirt of the catalyst carrier and the wall of the tube. Thus the temperature of the gas exiting the catalyst bed is measured. In this arrangement, the thermowell will extend through the seal on the top of each carrier.

In an alternate arrangement, the temperature measuring means, such as the thermowell, may be located in the centre of the annular container as illustrated in FIGS. 9 and 10. In this arrangement, the flow will be as indicated by the arrows in FIG. 10, which is the reverse of the direction shown by the arrows in FIGS. 7 and 8.

It will be understood that whilst the catalyst carrier has been described with particular reference to a use in a tube of circular cross-section the tube may be of non-circular cross-section for example, it may be a plate reactor. Where the tube is of non-circular cross-section, the carrier will be of the appropriate shape. In this arrangement, the annulus will not be a circular ring and this term should be construed accordingly.

The benefit of the present invention is illustrated in the graph of FIG. 11. Line X represents the peak temperature at the exit of each radial catalyst bed. As illustrated the tube includes 10 catalyst carriers. Line Y represents the temperature rise across the radial bed operating under adiabatic conditions while line Z represents the cooling of gas after the radial bed as it flows between the catalyst carrier and the reactor tube wall. It will therefore be seen that the peak temperature at the exit of each radial catalyst bed can be measured with high accuracy since it is measured at a point where no reaction occurs.

1. A reactor tube comprising a plurality of catalyst carriers, each of said catalyst carriers comprising:
an annular container for holding catalyst in use, said container having a perforated inner wall defining a tube, a perforated outer wall, a top surface closing the annular container and a bottom surface closing the annular container;
a surface closing the bottom of said tube formed by the inner wall of the annular container;
a skirt extending upwardly from the perforated outer wall of the annular container from a position at least one of at and near the bottom surface of said container, the skirt having a circumferential outer edge;
a seal located at or near the top surface and extending outward from the container beyond the circumferential outer edge of the skirt, the skirt extending upward to a position below the seal;
characterised in that the reactor tube additionally includes a temperature measuring device configured to measure the temperature in two or more of said catalyst carriers; said temperature measuring device being located at least one of between the catalyst carrier outer wall and the skirt, external of the skirt and in the interior space of the annular container.

2. The reactor tube according to claim 1 wherein the temperature measuring device measures the temperature in substantially the same position in each of the catalyst carriers in which temperature is to be measured.

3. (canceled)

4. (canceled)

5. The reactor tube according to claim 1 wherein the temperature measuring device is a multipoint thermocouple.

6. The reactor tube according to claim 1 wherein the catalyst carrier has a conical shaped structure through which in use the temperature measuring means may be inserted.

7. The reactor tube according to claim 1 additionally comprising catalyst in each carrier.

8. The reactor tube according to claim 7 wherein the catalyst is selected from the group consisting of a mercuric catalyst, a gold-based catalyst and a combination thereof for the production of vinyl chloride.

9. A reactor comprising at least one of the reactor tubes of claim 1.

10. A process for controlling a reaction, the process comprising the steps of:
supplying reactants to a reactor of claim 9 such that they come into contact with the catalyst in the carrier;
allowing the reaction to occur;
monitoring the temperature measured by the temperature measuring device; and
adjusting a parameter of the reactor when the temperature measured by the temperature measuring device deviates from a predetermined range.

11. The process according to claim 10 wherein the process is a reaction for the formation of vinyl chloride.

12. The process according to claim 10 wherein the parameter that is adjusted is at least one of the flow rate of one or more reactants, the temperature and the flow rate of the heat transfer medium.

13. A process for loading catalyst carriers into a reactor tube comprising inserting a temperature measuring means into the reactor tube and then loading the catalyst carriers over the temperature measuring means.

14. A process for loading catalyst carriers into a reactor tube comprising inserting the catalyst carriers into the reactor tube and then feeding ire a temperature measuring means into the carriers.
